(19) United States
(12) Patent Application Publication
(10) Pub. No.: US 2026/0144446 A1
(43) Pub. Date: May 28, 2026

(54) Apparatus For Detecting Bleeding At Patient Access Site

(71) Applicant: Freddy Junior West, Albany, GA (US)

(72) Inventor: Freddy Junior West, Albany, GA (US)

(21) Appl. No.: 18/831,335

(22) Filed: Nov. 22, 2024

(51) Int. Cl. A61B 5/02; A61B 5/00

(52) U.S. Cl. CPC A61B 5/02042; A61B 5/6833

(57) ABSTRACT

An apparatus for detecting bleeding at a patient access site includes a housing having an open bottom portion with a bottom surface, an open intermediate portion positioned above the open bottom portion and connected thereto forming a central opening, and a closed top portion attached to the intermediate portion capping the central opening. The housing also has a port adapted receive a tube for transporting blood. An output terminal is attached to the housing. A sensor is connected to the output terminal, attached to the housing and disposed in the central opening. The sensor is adapted to detect blood leaking from the patient access site and deliver a signal to the output terminal. An adhesive members attached to the bottom surface of the open bottom portion of the housing detachably attaches the housing about the patient access site.

### TECHNICAL FIELD OF THE INVENTION

This invention relates generally to hemodialysis medical procedures that use a needle at an arterial or venous patient access site. Such needles can dislodge during treatment causing blood to leak from the access site. The present invention provides an apparatus for the patient access site that inhibits needle dislodgement and detects blood loss due to needle dislodgement during dialysis treatment.

### BACKGROUND OF THE INVENTION

Each year, in the United States of America alone, over one hundred thousand people are initially diagnosed with kidney failure. There a currently well over half a million patients undergoing dialysis treatment for renal disease and near end stage kidney disease. Dialysis does not cure the kidney disease but can extend the life of the patient. The dialysis procedure involves removing blood from the patient and using a dialyzer, such as an artificial kidney or dialysis machine, to cleanse blood a diseased kidney can no longer cleanse effectively.

Large bore needles are used to remove a sufficient volume of blood from the patient to deliver to the dialysis machine and to return cleansed blood back to the patient. One large bore needle removes blood from the patient/and delivers the blood through tubing to the dialysis machine, while, another large bore needle delivers treated blood for the patient through tubing connected to the dialysis machine. It is not uncommon for a needle to dislodge at the patient access site especially when a patient moves, whether intentionally or accidentally. It is therefore desirable to detect bleeding and therefore needle dislodgement during dialysis and other procedures. It is also desirable to protect the needle and tubing at the patient access site to inhibit dislodgement.

U.S. Pat. No. 7,147,615 B1, entitled, Needle Dislodgement Detection, which issued Dec. 12, 2006 to Wariar et al., discloses methods and apparatuses for detecting, monitoring and/or controlling blood loss from a patient due to needle dislodgement. Wariar uses a sensor and a multi-piece sensor holder that overlies the sensor such that the sensor holder secures the sensor to the patient for detecting wetness due to blood loss from the patient upon dislodgement of the needle. The sensor holder includes a pad configuration such that it is sized to cover the needle and access region therein. The sensor holder includes a rigid material, such as a rigid plastic material that has a preferable dome shape. The sensor holder can act to shield and protect the sensor, needle and other components that it covers in addition to properly positioning and securing the sensor over the access or insertion region of the needle.

While the Wariar apparatus does detect blood leaks, it does so with a sensor positioned primarily above the needle and depends on enough blood to wet a pad of absorbent material. The absorbent material lies above the access site but below the sensor so that the sensor detects blood on the pad. Unfortunately, there can be bleeding without wetting the absorbent pad sufficiently enough to operate the sensor to trigger an alarm. Accordingly, it will be appreciated that it would be highly desirable to have an apparatus for detecting bleeding at a patient access site that does not depend on absorbent material lifting blood uphill against gravity. It is also desirable to have an apparatus of simple construction.

### SUMMARY OF THE INVENTION

An apparatus for detecting bleeding at a patient access site comprises a housing having an open bottom portion with a bottom surface, an open intermediate portion connected to and positioned above the open bottom portion forming a central opening, and a closed top portion attached to the intermediate portion capping the central opening. The housing also has a port adapted receive a blood transporting tube.

An output terminal is attached to the housing. A sensor is connected to the output terminal, attached to the housing and disposed in the central opening. The sensor is adapted to detect blood leaking from the patient access site and deliver a signal to the output terminal. An adhesive member attached to the bottom surface of the open bottom portion of the housing removably attaches the housing about the patient access site. When attached with the adhesive member, the housing forms a shield over the patient access site.

The first port and terminal are disposed on opposite distal end portions of the housing. When desirable or needed, the housing contains a second port adapted receive a second blood transporting tube. The ports are preferably parallel to one another and the sensor is preferably attached along the bottom portion of the housing.

These and other aspects, objects, features and advantages of the present invention will be more clearly understood and appreciated from a review of the following detailed description of the preferred embodiments and appended claims, and by reference to the accompanying drawing figures.

### BRIEF DESCRIPTION OF THE DRAWING FIGURES

FIG. 1 is a diagrammatic perspective top view of a preferred embodiment of a housing for an apparatus for detecting bleeding at a patient access site according to the present invention.

FIG. 2 is a diagrammatic bottom perspective bottom view of the housing of FIG. 1.

FIG. 3 is a diagrammatic top view of the housing of FIG. 1.

FIG. 4 is a diagrammatic side view of the housing of FIG. 1.

FIG. 5 is a diagrammatic sectional view taken along line 5-5 of the housing of FIG. 3.

FIG. 6 is a diagrammatic perspective top view of another preferred embodiment of a housing for an apparatus for detecting bleeding at a patient access site according to the present invention.

FIG. 7 is a diagrammatic perspective top view, similar to FIG. 6, but illustrating external battery placement.

FIG. 8 is a diagrammatic perspective bottom view, similar to FIG. 7, but illustrating another battery placement.

### DETAILED DESCRIPTION OF THE PRESENT INVENTION

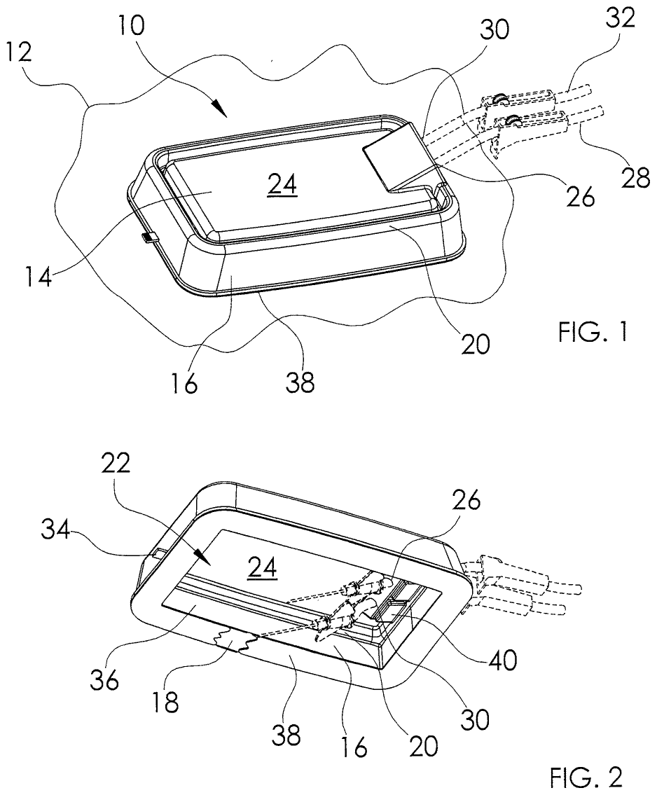

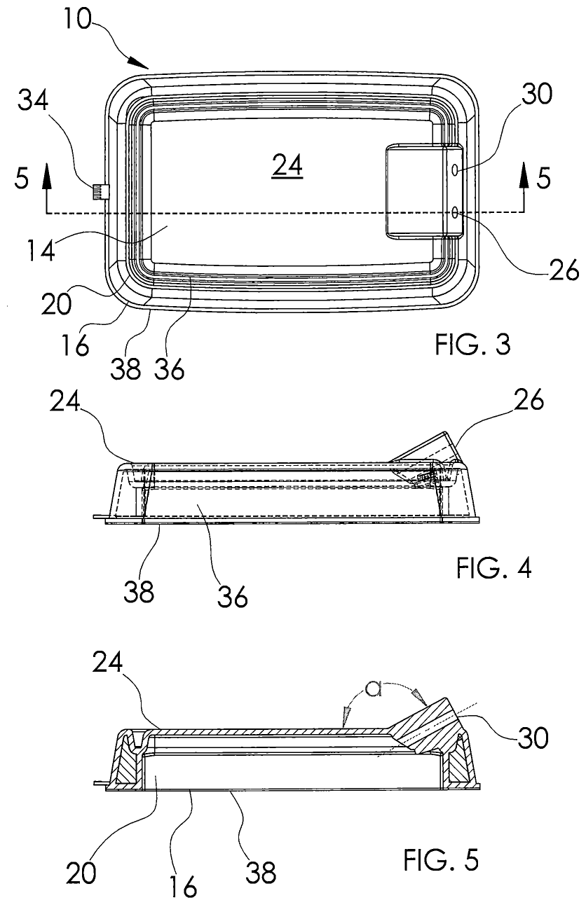

Referring to FIGS. 1-5, an apparatus 10 for detecting bleeding at a patient access site 12 includes a housing 14 having an open bottom portion 16 with a bottom surface 18, an open intermediate portion 20 positioned above the open bottom portion 16 and connected thereto forming a central opening 22. A closed top portion 24 is attached to the intermediate portion 20 of the housing 14 capping the central opening 22. The central opening 22 is larger near the open bottom portion 16 than open intermediate portion 20 and larger near intermediate portion 20 than closed top portion 24. Housing 14 thus has sloping or contoured sidewalls. Preferably, the bottom and intermediate portions 16, 20 of housing 14 form a step at their junction that provides strength to the structure without added material. This provides rigidity with less material thereby providing a more cost effective housing.

Housing 14 is preferably a unitary structure formed of a single material, such as injection molded polyurethane or other medical grade polymer. Plastic materials bonded together into appropriate unitary configurations can be used, as well as, laminated materials, paper and metal foils, but injection molding is generally more cost effective.

The housing 14 has a first port 26 adapted receive a first tube 28 for transporting blood in one of the directions to and from the patient access site; while a second port 30 is adapted receive a second tube 32 for transporting blood in the opposite of the other of the directions to and from the patient access site. Blood is transported from the patient via first transport tube 28 to a dialysis machine or other dialyzer as is well known in the art and returned via second transport tube 32. Each tube preferably has a connulation needle preferably outfitted with tabs or wings to hold the needle in position on the patient to help inhibit pullout after the needle is inserted under the skin.

An output terminal 34 is preferably attached to one end of the housing. First and second ports 26, 30 are preferably disposed on an end portion of the housing 14 distal from the terminal 34. Such disposition helps prevent entangling blood lines and electrical lines which may also connect to the dialysis machine.

A sensor 36 is connected to output terminal 34, attached to housing 14 and disposed in the central opening 22. Sensor 36 may be one of a variety of capacitive, magnet or resistive sensors used for medical detection purposes. Sensor 36 is adapted to detect blood leaking from access site 12 and deliver a signal to output terminal 34. Positioning sensor 36 along bottom portion 16 of housing 14 allows the sensor to detect blood virtually as soon as it leaks owing to its gravity feed. When in use, preferably on a prepared lower arm with the patient sitting upright or reclining, leaking blood will gravitate towards bottom portion 16 of housing 14 to the sensor. Placing sensor 36 in such location ensures earliest blood contact and therefore earliest indication of problematic needle dislodgement. Quite routinely, the patient's arm is prepared surgically to form a fistula which will provide adequate blood flow for a dialysis procedure.

An adhesive member 38 is fastened to the bottom surface 18 of open bottom portion 16 of housing 14 and is adapted to removably attach housing 14 about the patient access site 12. While the housing could simply be taped about the patient's arm with one of the adhesive or stretchy medical tapes available, using an incorporated adhesive member instead reduces handling and possible contamination to help maintain a more sterile environment for blood transfer. Adhesive member 38 preferably has a peel away cover (not shown) made of paper, plastic or the like to be discarded prior to attaching member 38 to the patient. Such peel away cover members are well known in the art to maintain sterility and adhesive integrity. Because a single treatment session can often routinely last three to four hours or more at a time, adhesive and stretchy medical tapes can irritate to the patient during the procedure thereby causing movement that can dislodge a needle. Gently applying adhesive member 38 to the skin causes minimal skin stretching or compression of the skin and is much less likely to cause discomfort that would lead to needle dislodgement.

When internal power is desired, a battery 40 can be used. Preferably, battery 40 is attached to the interior of the housing near output terminal 34. Alternatively, battery 40 may be attached to the exterior of the housing. Also, a remote power supply may be used via terminal 34. Ideally, terminal 34 connects sensor 36 to a dialysis machine either directly or via a controller that powers the sensor and an alarm, as is known in the art.

Still referring to FIGS. 1-5, each of the tube receiving ports 26, 28 are angled with respect to patient access site 12 and with respect to top surface 24 of housing 12 and bottom surface 18 as well. Top and bottom surfaces 24, 18 are generally parallel-to one another. Bottom surface 18 attaches to the patient access site 12 and is therefore parallel to it. Preferably, ports 26, 30 lie at an angle α between about 140° and 165° relative to top surface 18. At angles more than about 165° there is less resistance to horizontal tube pull out and thereby needle pull out. At angles less than about 140° there is less resistance to vertical tube pull out and thereby needle pull out. More preferably ports 28, 30 lie at an angle α about 152°relative to top surface 24. When tubes 28, 32 are inserted, this angle α permits the needles be easily inserted under the skin. When needles with hold down wings are inserted, the housing covers them to help prevent needle pull out dislodgement.

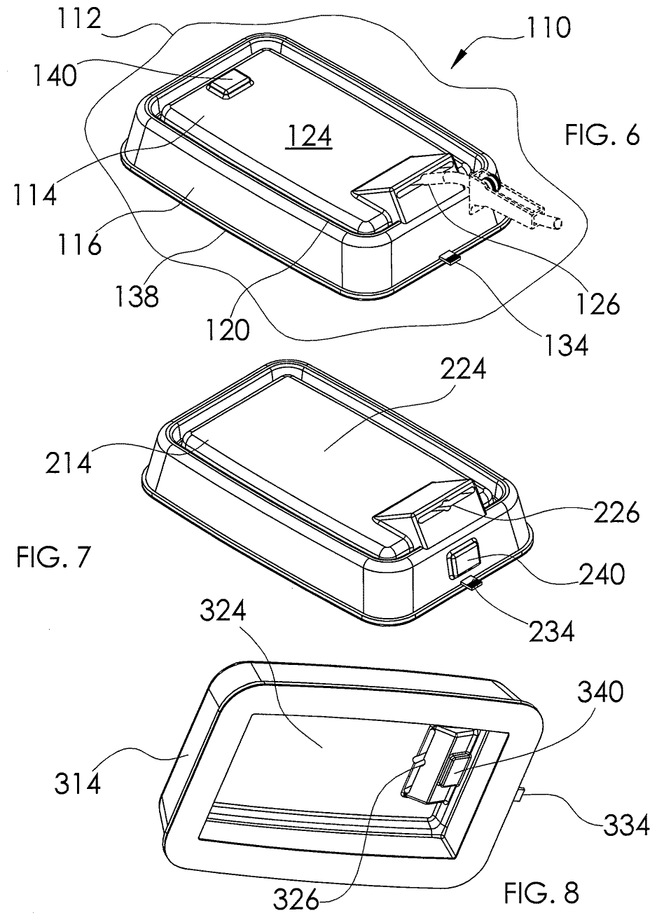

Referring now to FIG. 6 an apparatus 110 for detecting bleeding at a patient access site 112 includes a housing 114 having an open bottom portion 116 with a bottom surface, an open intermediate portion 120 positioned above the open bottom portion 116 and connected thereto forming a central interior opening. A closed top portion 124 is attached to the intermediate portion 120 of the housing 114 capping the central opening. Housing 114 defines a port 126 adapted receive a tube for transporting blood between the patient access site and dialysis machine. Port 126 has a central circular opening with an enlongated slot on each side. The slots and central circular opening are designed to receive a tube and needle with wings so that the wings can help secure the needle at the patient access site. Port 126 is similar in function to port 26 of FIG. 1 in that it accommodates tubing for transporting blood during the medical procedure. The difference is that a pair of housings 114 are used where patient access exists in dual sites which is sometimes the case due to the physical attributes and condition of the patient. With dual sites, one blood transport tube removes contaminated blood from one patient access site through one housing and delivers it to the dialysis machine, while the other blood transport tube returns cleansed blood from the dialysis machine through the other housing to the other access site.

An output terminal 134 is preferably attached to one end of the housing 114. In the embodiment of the invention shown in FIG. 6, port 126 is disposed on the same end portion of housing 114 as terminal 134. Also, a battery 140 and port 126 may be disposed on the exterior of the housing, conveniently located to power a sensor.

Referring now to FIG. 7, a battery 240 may be conveniently located on the end of the housing 214, between port 226 and terminal 234. Housing 214 defines a port 226 adapted receive a tube for transporting blood between the patient access site and dialysis machine. Port 226 has a central circular opening with an enlongated slot on each side. The slots and central circular opening are designed to receive a tube and needle with wings so that the wings can help secure the needle at the patient access site. Port 226 is similar in function to port 26 of FIG. 1 in that it accommodates tubing for transporting blood during the medical procedure. The difference is that a pair of housings 214 are used where patient access exists in dual sites which is sometimes the case due to the physical attributes and condition of the patient. With dual sites, one blood transport tube removes contaminated blood from one patient access site through the housing and delivers it to the dialysis machine, while the other blood transport tube returns cleansed blood from the dialysis machine thorough the housing to the other patient access site.

Referring now to FIG. 8, housing 314 defines a port 326 adapted receive a tube for transporting blood between the patient access site and dialysis machine. Port 326 has a central circular opening with an enlongated slot on each side. The slots and central circular opening are designed to receive a tube and needle with wings so that the wings can help secure the needle at the patient access site. Port 326 is similar in function to port 26 of FIG. 1 in that it accommodates tubing for transporting blood during the medical procedure. The difference is that a pair of housings 314 are required where patient access exists in dual sites which is sometimes the case due to the physical condition of the patient. With dual sites, one blood transport tube removes contaminated blood from one access site and delivers it to the dialysis machine, while the other blood transport tube returns cleansed blood from the dialysis machine to the other access site. A battery 340 may be conveniently located on the interior of the housing 314 between port 326 and terminal 334.

### Mode of Operation of the Invention

Operation of the present invention is believed to be apparent from the foregoing detailed description and appended drawings, but a few words will be added for emphasis. Prior to use, both the patient access site and the apparatus for detecting bleeding at the patient access site require preparation. The access site is preferably cleansed and sterilized in the normal manner with the patient sitting upright or reclining, The apparatus is readied by feeding the blood transporting tubes through the ports in the housing, if they do not come preinstalled. The blood transporting tubes are connected the dialysis machine which receives the contaminated blood, treats the blood and returns cleansed blood back to the patient.

Where a battery is used, it is readied for use at this time. If there is no battery, then electrical leads are attached to the output terminal. In either case, the sensor is energized and ready for use. The needles are uncovered and inserted under the skin of the patient at the access site. Now, the peel away member is removed and the housing is pressed onto the access site. Leaking blood will gravitate towards bottom portion the of housing. Placing the sensor in such location ensures earliest blood contact and therefore earliest indication of problematic needle dislodgement.

Because treatment may routinely last two to four hours at a time, adhesive and stretchy medical tapes often irritate the patient during the procedure causing movement that may dislodge the needles. Gently applying the adhesive member to the skin provides good adhesion while causing minimal stretching or compression of the skin thereby reducing discomfort that would lead to needle dislodgement.

It can now be appreciated that an apparatus for detecting bleeding at a patient access sire has been presented that not only covers the patient access site but detects blood loss at the site as well. While the invention has been described with particular reference to the preferred embodiments, it will be understood by those skilled in the art that various changes may be made and equivalents may be substituted for elements of the preferred embodiments without departing from the invention. For example, while the housing shield is depicted with a stepped configuration that provides internal surfaces, spaces and locations for the sensor on the bottom step, the step need not form sharp corners. The step can slope or have curves depending on how it was formed. And while specific battery placements have been described, the exact placement on or in the housing is not critical. The important factor is that it be unobtrusive so as to not interfere with other elements of the apparatus. It is accordingly intended that the claims shall cover all such modifications and applications as do not depart from the true spirit and scope of the invention.

The invention claimed is:

1. An apparatus for detecting bleeding at a patient access site, comprising:
a housing having an open bottom portion with a bottom surface, an open intermediate portion positioned above said open bottom portion and connected thereto forming a central opening, and a closed top portion attached to said intermediate portion capping said central opening; said housing having a port adapted receive a tube for transporting blood;
an output terminal attached to said housing; and
a sensor connected to said output terminal, attached to said housing and disposed in said central opening, said sensor adapted to detect blood leaking from said patient access site and to deliver a signal to said output terminal.

2. The apparatus for detecting bleeding, as set forth in claim 1 including an adhesive member attached to said bottom surface of said open bottom portion of said housing and adapted to removably attach said housing about said patient access site.

3. The apparatus for detecting bleeding, as set forth in claim 1, wherein said housing is adapted to cover said patient access site forming a shield over said patient access site.

4. The apparatus for detecting bleeding, as set forth in claim 1, wherein said housing is formed of a single piece of material.

5. The apparatus for detecting bleeding, as set forth in claim 1, wherein said port and terminal are disposed on opposite distal portions of said housing.

6. The apparatus for detecting bleeding, as set forth in claim 1 wherein said port lies at an angle in a range of about 140 degrees to 165 degrees relative to said top portion of said housing.

7. The apparatus for detecting bleeding, as set forth in claim 6, wherein said port lies at an angle of approximately 152 degrees relative to said top portion of said housing.

8. The apparatus for detecting bleeding, as set forth in claim 1, wherein said housing includes a second port formed in said housing and extending in said housing approximately parallel to said first port.

9. The apparatus for detecting bleeding, as set forth in claim 1, wherein said housing defines a second port, said second port being adapted to receive a second tuber for transporting blood.

10. The apparatus for detecting bleeding, as set forth in claim 2, wherein said sensor is attached along said bottom portion of said housing adjacent and above said adhesive member so that blood present inside said housing gravitates toward said sensor.

11. The apparatus for detecting bleeding, as set forth in claim 1, including a power source for said sensor.

12. The apparatus for detecting bleeding, as set forth in claim 11, wherein said power source is a battery.

13. The apparatus for detecting bleeding, as set forth in claim 12, wherein said battery is disposed in said central opening of said housing.

14. The apparatus. for detecting bleeding, as set forth in claim 13 wherein said battery is positioned adjacent said port.

15. The apparatus for detecting bleeding, as set forth in claim 12, wherein said battery is disposed outside of said housing.

16. The apparatus for detecting bleeding, as set forth in claim 12, wherein said battery is electrically connected to said sensor via said output terminal.

17. The apparatus for detecting bleeding, as set forth in claim 1: wherein said housing is formed of a single piece of material and adapted to cover said patient access site forming a shield over said patient access site;
said port lies at an angle in a range of about 140 degrees to 165 degrees relative to said top portion of said housing; and
including an adhesive member attached to said bottom surface of said open bottom portion of said housing and adapted to removably attach said housing about said patient access site.

18. An apparatus for detecting bleeding at a patient access site, as set forth in claim 1 wherein
said port lies at an angle in a range of about 140 degrees to 165 degrees relative to said top portion of said housing, and
including an adhesive member attached to said bottom surface of said open bottom portion of said housing and adapted to removably attach said housing about said patient access site.

19. An apparatus for detecting bleeding at a patient access site, as set forth in claim 1 wherein
said housing is formed of a single piece of material adapted to cover said patient access site forming a shield over said patient access site; and
said port in said housing said port lies at an angle of approximately 152 degrees relative to said top portion of said housing.

20. An apparatus for detecting bleeding at a patient access site, comprising:
a housing having an open bottom portion with a bottom surface, an open intermediate portion positioned above said open bottom portion and connected thereto forming a central opening, and a closed top portion attached to said intermediate portion capping said central opening; said housing having a port adapted receive a tube for transporting blood,-said port being disposed at an angle in a range of about 140 degrees to 165 degrees relative to said top portion of said housing; said housing being adapted to cover said patient access site forming a shield over said patient access site. an output terminal attached to said housing;
a sensor connected to said output terminal, attached to said housing and disposed in said central opening, said sensor being adapted to detect blood leaking from said patient access site and to deliver a signal to said output terminal;
a power source for said sensor; and
an adhesive member attached to said bottom surface of said open bottom portion of said housing and adapted to removably attach said housing about said patient access site, said sensor being attached along said bottom portion of said housing adjacent and above said adhesive member so that blood present inside said housing gravitates toward said sensor.
